(19) United States
(12) Patent Application Publication
(10) Pub. No.: US 2012/0172824 A1
(43) Pub. Date: Jul. 5, 2012

(54) Caring aide

(76) Inventors: Abdikarim Khaknazarov, Brooklyn, NY (US); Kanaatoy Solieva, Brooklyn, NY (US)

(21) Appl. No.: 12/930,283

(22) Filed: Jan. 3, 2011

(51) Int. Cl. A61F 13/42

(52) U.S. Cl. 604/361

(57) ABSTRACT

Working as HHA and babysitters we have faced the problem that most children and patients don't realize their urinating. In order to provide timely replacement of pampers we have decided to create a system that would give a signal that pampers are wet. Our system called Caring Aide comprises a tiny battery, a hydro switch, electric wires, signaling device, and an electronic transmitter. When a hydro switch adjoins to the moisture (urine), the signaling device starts to work: the light blinks or the music sounds. A signaling device can be installed in different parts of diapers or in any other place where care providers are. This device will help children and old people avoid getting wet especially at night time. It can be used at care of bed patients as it will promote avoidance decubitus or sore skin. Caring Aide fits for any type of diapers.

### DESCRIPTION OF THE INVENTION

Purpose

My caring aide is used to help in the caring practice of children and the elderly. If the diapers and pampers have an electronic transmitter which signals about the wet, we will help children and the elderly to keep their health and save time of parents and health care providers.

### Drawings

My invention and how it attaches to a diapers, pampers, and underwear are shown in FIGURES

A with variations 1, 1′, 2, 3, 3′.

B with variations 4,5,6.

Every figure has a hydro switch—GS, a battery—B, and Signaling Device—SD; FIGS. 4,5,6 include also electronic transmitter T+GS,B.

FIG. 1 includes one blinking light and one ringtone.

FIG. 1′ includes one blinking light and one ringtone, named “Diaper duty” that can be worn without cloths on indoors.

FIG. 2 includes two blinking lights.

FIG. 3 has two ringtones at the front and back side.

FIG. 3′—two ringtones. The blinking lights or ringtones can be replaced on different places of diapers.

FIG. 4 includes an electronic transmitter that transfers signal to a signaling device.

FIG. 5—an electronic transmitter that transfers signal to a signaling device that can be portable, to be even in the pocket of care workers.

FIG. 6.—two electronic transmitters (at the front and back side) that transfer signal to another room where parents and babysitters are.

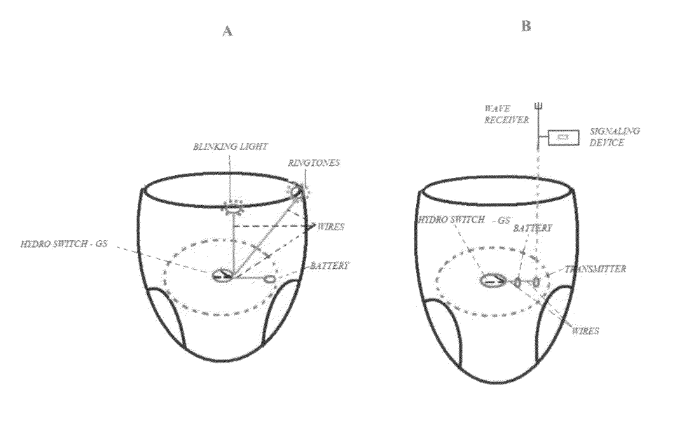

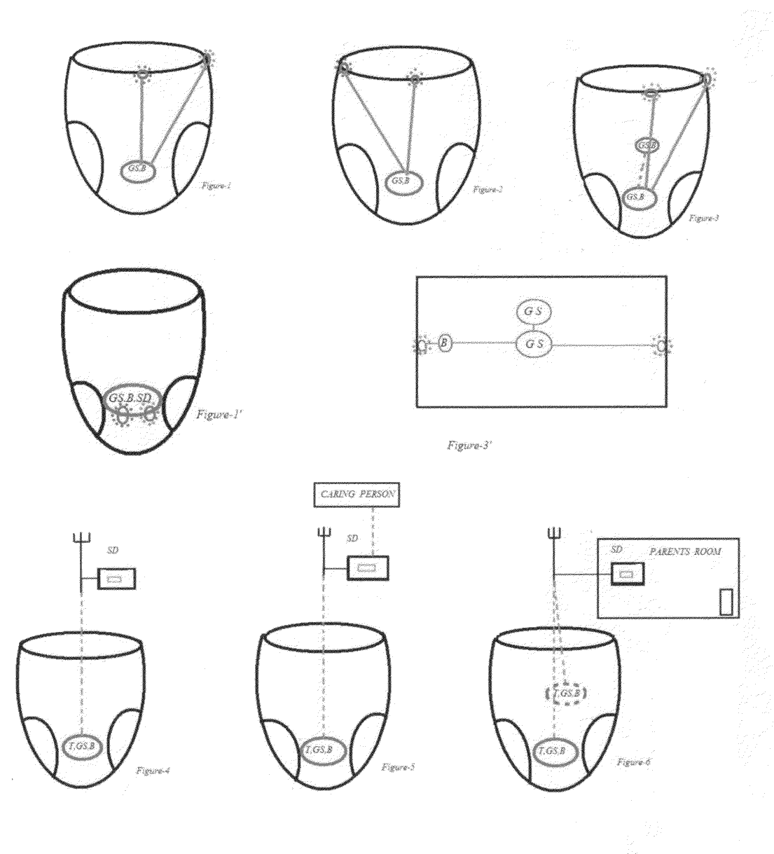

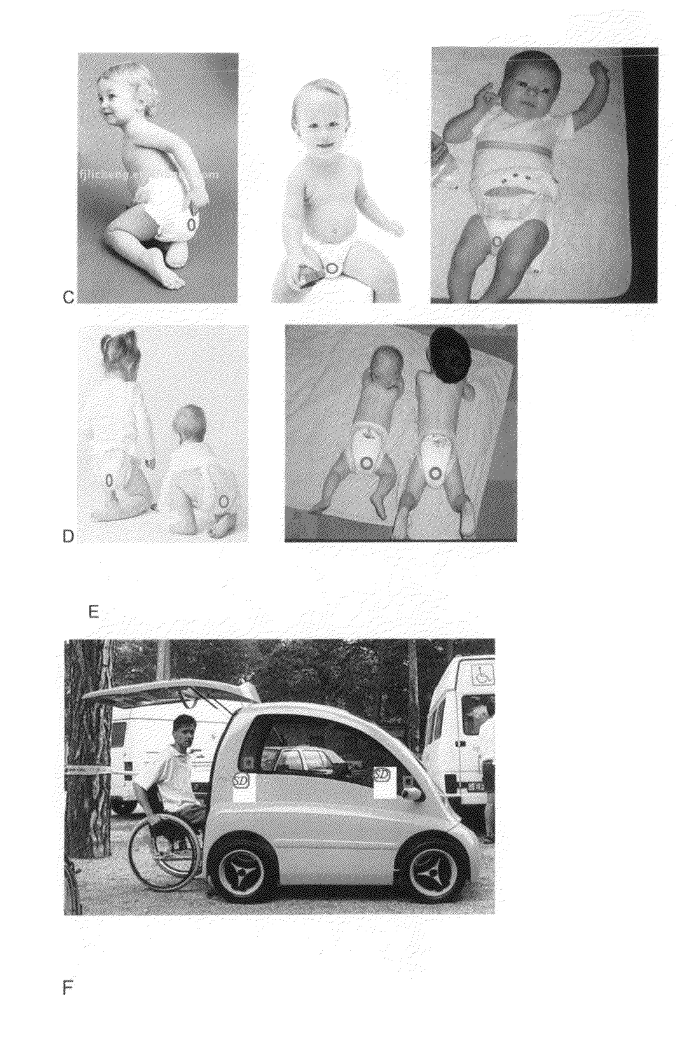

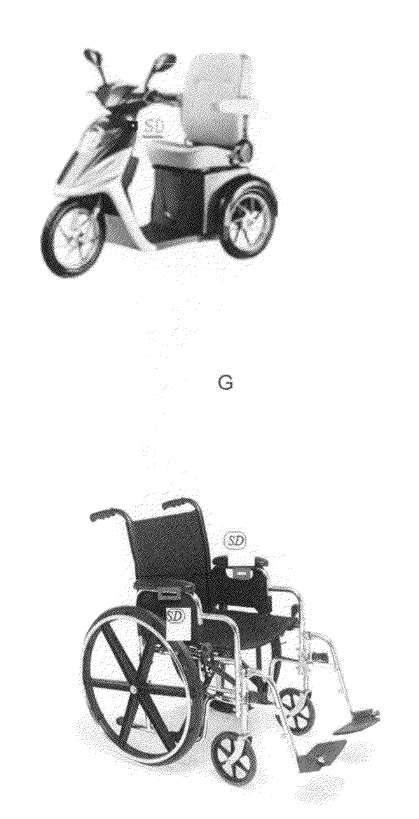

### DESCRIPTION OF THE PARTS

My caring aide is for use with any type of diapers, pampers, underwear, and pads. The caring aid has two versions.

The first version—A (with variations 1, 1′, 2, 3, 3′)—includes:

1. The tiny battery;

2. The hydro switch turns the system on, when the pampers is wet;

3. The electric wires carrying electric current to the signaling device;

4. The signaling device (blinking light or ringtones).

The second version—B (with variations 4,5,6)—includes:

1. The tiny battery;

2. The hydro switch transmitting electric current from the battery to transmitter of electric waves;

3. The electronic transmitter transferring electric waves to the signaling device;

4. The receiver of electric waves;

5. The signaling device (blinking light or ringtones).

Use

Working principle of my system is based on the following:

A. The tiny battery providing the system with a current and the hydro switch are located in the front side of pampers where they have absorbent on that place where urine usually flows down. When the hydro switch is adjacent to the moisture (urine), it connects a current to system through the electric wires installed in the front side or sideways from the urinating place till top sides of diapers. The electric wires connecting with the hydro switch carries electric current to the signaling device. Then the signaling device starts to work: the light blinks or the music sounds.

B. The tiny battery providing the system with a current and the hydro switch are located in the front side of pampers where they have absorbent on that place where urine usually flows down. When the hydro switch is adjacent to the moisture (urine), current from the battery through the switch goes to the transmitter that transfers electric waves to a receiver/signaling device in another room (it can be the phone), where the parents or babysitters are.

Novel Features

I had looked in the pharmacies, medical supplies, stores and in various medical supply catalogs and could nowhere find a product similar to mine. I did not find any device that could be attached to the diapers and pampers (or underwear) that would give the information on urine and excrement (feces) receipt. In many cases the elderly and children especially at night time lie in urine or are even worse in an excrement without supervision. I believe that my invention has the following novel features which are not disclosed in the prior art:

a) When the child is wet, then the signaling device automatically works; Photos-C,D

b) Warns about urine and excrement;

c) Promotes rendering of the immediate help to sick, old people and children;

d) It is very convenient to use at care of bed patients as it will promote avoidance decubitus or sore skin;

e) The second version B includes improbable possibilities of use of the electronic transmitter transferring electric waves to the signaling device (if can be the phone) by parents, babysitters, HHA, and the medical staff. Being in other room they can hear a signal going from a signaling device;

f) The second version B provides great opportunities, especially at night when children and elderly people are wet with urine or excrement it is censored by hydro-switches (or hydro-sensors) and automatically turns on the signal system through the electric transmitter and lets change pampers as well as diapers of a baby or patient on time.

g) There is a possibility of installation of signaling device in cars for invalids; Photo—E

h) There is a possibility of installation of signaling device in wheelchairs, auto wheelchairs and airplanes. Photos—F,G

Advantages

My invention fits for all types of pampers, diapers, and underwear. Its installation is very useful for children, sick and old people because it helps avoid getting wet, sick, and decubitus or sore skin, especially it is useful for patients with diabetes. It helps save people's health and financial resources.

Claim

Caring Aide consisting of a hydro switch—GS, a battery—B, Signaling Device—SD, and an electronic transmitter—T can be attached to all types of the diapers and pampers (or underwear). It gives a sign about the wet when a hydro switch is adjacent to the moisture (urine). Caring Aide has two versions with many variations. The first type works locally, SD is arranged in diapers, while the second type can be portable or connected with the transmission signal at a distance—in another room where the parents, babysitters or care providers.

Abstract of the Disclosure

The caring aide is suitable for all types of pampers, diapers, and underwear. It has two types with variations. The first one (A) is equipped with a tiny battery—power source, a hydro switch, electric wires, and a signaling device. The tiny battery and the hydro switch are installed in pampers where they have absorbent on that place where urine flows down. When the hydro switch adjoins to the moisture (urine), it connects a current to system through the electric wires. The signaling device starts to work: the light blinks or the music sounds. The second type (B) includes a tiny battery, a hydro switch, a transmitter of electric waves, and a signaling device. When the hydro switch is adjacent to moisture, current from the battery through the switch goes to the transmitter that transfers electric waves to a receiver/signaling device in another room, where the parents or babysitters are.

Motivation of the use of color drawings and photos

Color drawings and photos were used for the most precise representation of our proposed system, also showing the location of the wire and the selector lever

Testing Results

Effectiveness of my device I tested on my practice as HHA. From Jan. 15, 2009 till Apr. 15, 2010 I have been working as HHA in Williamsburg with Mendel Haight and worked with a boy named Abror Mirobidov at the same time. My daughters have been baby sitting with Milda Su Seyhan on weekends. I tested a prototype version of my device which was set in the diapers, pampers, and underwear. I figured out that with help of my device you can save the health of children and old people. When the diaper or pampers gets wet it will ring or blink to let other people know and change it on time. Health of the person is the most important fact of the life.

We claim:

1. Caring Aide consisting of a hydro switch, a battery, electric wires, Signaling Device, and an electronic transmitter gives a sign about the wet (the light blinks or the music sounds) when a hydro switch is adjacent to the moisture (urine).

2. Caring Aide defined in claim 1 wherein the components range forming two versions with many variations:
a) the first version including the components said in claim 1 except electronic transmitter works locally since Signaling Device arranged in diapers; the variations are connected with the number and location of the blinking lights or ringtones;
b) Signaling Device of the second version comprising all components said in claim 1 can be portable or connected with the transmission signal at a distance—in another room where the parents, babysitters or care providers are; the variations are due to the number of electronic transmitter and location of Signaling Device.

3. Caring Aide defined in claim 1 can be attached to all types of the diapers, pampers, pads, and underwear.
